(19) United States
(12) Patent Application Publication
(10) Pub. No.: US 2011/0257186 A1
(43) Pub. Date: Oct. 20, 2011

(54) COMPOSITIONS AND METHODS FOR TREATING VISUAL DISORDERS

(76) Inventors: Ursula V. Staubli, Laguna Beach, CA (US); Yong-Xin Li, Mission Viejo, CA (US); Alan C. Foster, San Diego, CA (US)

(21) Appl. No.: 13/085,241

(22) Filed: Apr. 12, 2011

Related U.S. Application Data
(60) Provisional application No. 61/324,632, filed on Apr. 15, 2010.

(51) Int. Cl. A61K 31/5025; A61P 27/06; A61P 27/02

(52) U.S. Cl. 514/248

(57) ABSTRACT

Disclosed are methods of treating disorders of the eye by administering to a patient in need of such treatment a compound represented by the formula

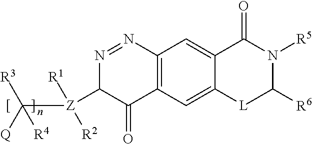

Wherein R¹ and R² are independently hydrogen, alkyl, substituted alkyl, cycloalkyl, alkynyl, substituted alkynyl, cyano, alkoxy, carboxamido, substituted carboxamido, and if R¹ and R² are alkyl, R¹ and R² may be joined with a bond or —(CH₂)ₘ— to produce a cycloalkyl, R³ and R⁴ are independently hydrogen, alkyl, hydroxyl, alkoxy, cyano, fluoro, and if R³ and R⁴ are alkyl, R³ and R⁴ may be joined with a bond or —(CH₂)ₘ— to produce a cycloalkyl, Q may be absent, hydrogen, alkyl, cycloalkyl, cycloalkenyl, alkoxy, substituted alkoxy, substituted thio, cyano, thionitrile, sulfonamide, substituted sulfonamide, substituted sulfonyl, aromatic, substituted aromatic, heteroaromatic, substituted heteroaromatic, or bicycloheteroaromatic, R⁵ is hydrogen, alkyl, cycloalkyl, or when R⁶ is also alkyl, together with R⁶ may form a heterocycloalkyl ring, R⁶ may be hydrogen, alkyl, substituted alkyl, or —OR⁷, R⁷ is alkyl or, when R⁵ is alkyl, together with R⁵ forms a 5-, 6-, or 7-membered ring, L may be —O—, —S—, —N═ or absent, Z may be carbon or nitrogen or absent, m is 1, 2 or 3, n is 0, 1 or 2, and when n is 0, Q may be directly bonded to Z, or a pharmaceutically acceptable addition salt of an acid or base thereof.

### CROSS REFERENCE TO RELATED APPLICATIONS

This patent application claims the benefit of U.S. Provisional Patent Application No. 61/324,632, which was filed on Apr. 15, 2010 and is hereby incorporated by reference in its entirety.

### 1. FIELD OF THE INVENTION

Disclosed herein is a method of treating disorders of the eye by administering to a patient in need of such treatment certain 3-Substituted-[1,2,3]-Benzotriazinone compounds.

### 2. BACKGROUND OF THE ART

Ampakines are a class of compounds known to enhance attention span and alertness, and facilitate learning and memory. The ampakines take their name from the glutamatergic AMPA receptor with which they strongly interact. The AMPA receptor, in turn, gets its name from AMPA, which selectively binds to it.

Unlike earlier stimulants (e.g. caffeine, methylphenidate (Ritalin), and the amphetamines), ampakines do not seem to have unpleasant, long-lasting side effects such as sleeplessness.

They are currently being investigated as potential treatment for a range of conditions involving mental disability and disturbances such as Alzheimer's disease, Parkinson's disease, schizophrenia, Treatment-resistant depression (TRD) or neurological disorders such as Attention Deficit Hyperactivity Disorder (ADHD), among others.

Ampakine activity has been established as one of the modes of action of the well-established class of nootropics, the racetam drugs such as piracetam, aniracetam, oxiracetam and pramiracetam, however these drugs have multiple modes of action and produce only weak AMPA receptor activation, and it is unclear how significant their ampakine actions are in producing their nootropic effects. More recently developed ampakine compounds are much more potent and selective for the AMPA receptor target.

US Patent Application No. 2010/0041647, which is hereby incorporated by reference in its entirety, discloses 3-Substituted-[1,2,3]-Benzotriazinone compounds for enhancing glutamatergic synaptic responses.

### BRIEF SUMMARY OF THE INVENTION

The present invention provides a method for treating a disorder of the eye, the method comprising the step of administering to a patient in need of said treatment a compound represented by the formula

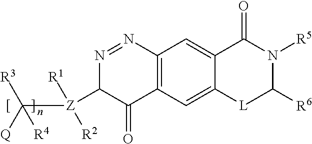

Wherein R¹ and R² are independently hydrogen, alkyl, substituted alkyl, cycloalkyl, alkynyl, substituted alkynyl, cyano, alkoxy, carboxamido, substituted carboxamido, and if R¹ and R² are alkyl, R¹ and R² may be joined with a bond or —(CH₂)ₘ— to produce a cycloalkyl, R³ and R⁴ are independently hydrogen, alkyl, hydroxyl, alkoxy, cyano, fluoro, and if R³ and R⁴ are alkyl, R³ and R⁴ may be joined with a bond or —(CH₂)ₘ— to produce a cycloalkyl, Q may be absent, hydrogen, alkyl, cycloalkyl, cycloalkenyl, alkoxy, substituted alkoxy, substituted thio, cyano, thionitrile, sulfonamide, substituted sulfonamide, substituted sulfonyl, aromatic, substituted aromatic, heteroaromatic, substituted heteroaromatic, or bicycloheteroaromatic, R⁵ is hydrogen, alkyl, cycloalkyl, or when R⁶ is also alkyl, together with R⁶ may form a heterocycloalkyl ring, R⁶ may be hydrogen, alkyl, substituted alkyl, or —OR⁷, R⁷ is alkyl or, when R⁵ is alkyl, together with R⁵ forms a 5-, 6-, or 7-membered ring, L may be —O—, —S—, —N═ or absent, Z may be carbon or nitrogen or absent, m is 1, 2 or 3, n is 0, 1 or 2, and when n is 0, Q may be directly bonded to Z; or a pharmaceutically acceptable addition salt thereof.

### BRIEF DESCRIPTION OF THE DRAWING

FIG. 1 shows a dose-dependent facilitation of long-term potentiation in rat visual cortex by the compound, benzo[1,2,5]oxadiazol-5-yl-piperidin-1-yl-methanone.

### DETAILED DESCRIPTION OF THE INVENTION

The method of the invention comprises administering to a patient a compound

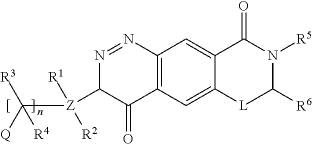

wherein R¹, R², R³, R⁴, R⁵, R⁶, R⁷, L, Q, Z, m and n are as defined above.
Preferably, in the compounds represented by the above formula, when R⁵ and R⁶ together form a morpholino ring and L is absent, then neither R¹, nor R² may be alkynlyl; or in the compounds of the formula when R⁵ is cyclopropyl, R¹, R², R³, R⁴, and R⁶ may not all be hydrogen, or Q may not be meta-fluorophenyl,

The above compounds may be used to treat a patient suffering from disorders of the eye.

To “treat,” as used here, means to deal with medically. It includes, for example, administering one or more of the above compounds to prevent the onset of a disorder, to alleviate its severity, and to prevent its reoccurrence.

Disorders which may be treated with the above compounds include macular edema, dry and wet macular degeneration, choroidal neovascularization, diabetic retinopathy, acute macular neuroretinopathy, central serous chorioretinopathy, cystoid macular edema, and diabetic macular edema, uveitis, retinitis, choroiditis, acute multifocal placoid pigment epitheliopathy, Behcet's disease, birdshot retinochoroidopathy, syphilis, lyme, tuberculosis, toxoplasmosis, intermediate uveitis (pars planitis), multifocal choroiditis, multiple evanescent white dot syndrome (mewds), ocular sarcoidosis, posterior scleritis, serpiginous choroiditis, subretinal fibrosis and uveitis syndrome, Vogt-Koyanagi-and Harada syndrome; retinal arterial occlusive disease, anterior uveitis, retinal vein occlusion, central retinal vein occlusion, disseminated intravascular coagulopathy, branch retinal vein occlusion, hypertensive fundus changes, ocular ischemic syndrome, retinal arterial microaneurysms, Coat's disease, parafoveal telangiectasis, hemiretinal vein occlusion, papillophlebitis, central retinal artery occlusion, branch retinal artery occlusion, carotid artery disease (CAD), frosted branch angiitis, sickle cell retinopathy, angioid streaks, familial exudative vitreoretinopathy, and Eales disease; traumatic/surgical conditions such as sympathetic ophthalmia, uveitic retinal disease, retinal detachment, trauma, photocoagulation, hypoperfusion during surgery, radiation retinopathy, and bone marrow transplant retinopathy; proliferative vitreal retinopathy and epiretinal membranes, and proliferative diabetic retinopathy; infectious disorders such as ocular histoplasmosis, ocular toxocariasis, presumed ocular histoplasmosis syndrome (PONS), endophthalmitis, toxoplasmosis, retinal diseases associated with HIV infection, choroidal disease associate with HIV infection, uveitic disease associate with HIV infection, viral retinitis, acute retinal necrosis, progressive outer retinal necrosis, fungal retinal diseases, ocular syphilis, ocular tuberculosis, diffuse unilateral subacute neuroretinitis, and myiasis; genetic disorders such as retinitis pigmentosa, systemic disorders with associated retinal dystrophies, congenital stationary night blindness, cone dystrophies, Stargardt's disease and fundus flavimaculatus, Best's disease, pattern dystrophy of the retinal pigmented epithelium, X-linked retinoschisis, Sorsby's fundus dystrophy, benign concentric maculopathy, Bietti's crystalline dystrophy, and pseudoxanthoma elasticum; retinal tears/holes such as retinal detachment, macular hole, and giant retinal tear; tumors such as retinal disease associated with tumors, congenital hypertrophy of the retinal pigmented epithelium, posterior uveal melanoma, choroidal hemangioma, choroidal osteoma, choroidal metastasis, combined hamartoma of the retina and retinal pigmented epithelium, retinoblastoma, vasoproliferative tumors of the ocular fundus, retinal astrocytoma, and intraocular lymphoid tumors; punctate inner choroidopathy, acute posterior multifocal placoid pigment epitheliopathy, myopic retinal degeneration, acute retinal pigment epitheliitis, retinitis pigmentosa, proliferative vitreal retinopathy (PVR), age-related macular degeneration (ARMD), diabetic retinopathy, diabetic macular edema, retinal detachment, retinal tear, uveitus, cytomegalovirus retinitis, and glaucoma.

The above compounds may be used to enhance the induction of long-term potentiation in the primary visual cortex (V1). V1 is one synapse removed from the eye and is essential for decoding, processing and transforming visual inputs originating from the retina. Visual disorders mediated by V1 include, but are not limited to, amblyopia, stroke-induced blindness, visual dysfunction in Parkinson's disease and Alzheimer's disease, seizure-induced cortical blindness, induced visual dysfunction, and epileptic blindness.

The visual cortex integrates visual signals generated by the retina, and proper visual cortical function is necessary for normal vision. Amblyopia is defined as poor or indistinct vision by an eye that is physically normal. Amblyopia can be initiated by poor transmission of the visual image to the visual cortex during childhood. Abnormal visual processing may be caused by form deprivation (i.e. cataracts), anisomometropia (different retinal image size, or magnification, in each eye), or suppression resulting from strabismus (misalignment of the eyes). A prolonged transmission of poor quality visual images induces in a physiological change within the visual cortex that alters the perception within the visual cortex. Briefly, the visual cortex will “ignore” the poor vision from one eye. Hence amblyopics often lack visual acuity and stereopsis.

The above compounds may be administered at pharmaceutically effective amounts. Such amounts are normally the minimum dose necessary to achieve the desired therapeutic effect. The actual amount of the compound to be administered in any given case will be determined by a physician taking into account the relevant circumstances, such as the severity of the ophthalmic condition, the age and weight of the patient, the patient's general physical condition, and the route of administration.

The patient may be given the above compounds orally or by local delivery to the eye. Local delivery includes topical delivery, in which an ophthalmically acceptable formulation is instilled in the eye via an eye dropper or other applicator, delivery by injection into the eye, or delivery by a drug delivery system that is implanted into the eye or eye lid and releases drug over a period of time.

The above compounds are administered in combination with an ophthalmically-acceptable carrier or vehicle.

A composition which is ophthalmically acceptable is formulated such that it can be administered topically to the eye. The comfort should be maximized as much as possible, although sometimes formulation considerations (e.g. drug stability) may necessitate less than optimal comfort. In the case that comfort cannot be maximized, the liquid should be formulated such that the liquid is tolerable to the patient for topical ophthalmic use. Additionally, an ophthalmically acceptable liquid should either be packaged for single use, or contain a preservative to prevent contamination over multiple uses.

For ophthalmic application, solutions or medicaments are often prepared using a physiological saline solution as a major vehicle. Ophthalmic solutions are often maintained at a comfortable pH with an appropriate buffer system. The formulations may also contain conventional, pharmaceutically acceptable preservatives, stabilizers and surfactants.

Various buffers and means for adjusting pH may be used so long as the resulting preparation is ophthalmically acceptable. Accordingly, buffers include, but are not limited to, acetate buffers, citrate buffers, phosphate buffers and borate buffers. Acids or bases may be used to adjust the pH of these formulations as needed.

Preservatives that may be used in the pharmaceutical compositions disclosed herein include, but are not limited to, benzalkonium chloride, chlorobutanol, thimerosal, phenylmercuric acetate and phenylmercuric nitrate.

Certain compositions contain solubility enhancing components (SECs) in amounts effective to enhance the solubility of the above compounds at a given pH. These SECs may be anionic in nature, and can be polymeric in nature. In one embodiment the SEC is a cellulose derivative, in another embodiment the SEC is not a cellulose derivative or a cyclodextrin. In these compositions, the SEC is used to enhance the solubility of the compound. In other words, in two compositions containing the compound which are identical except for the presence of an effective amount of the SEC, more compound will be dissolved in the composition containing the SEC than the in the composition not containing the SEC.

The SEC may include a non-ionic or polyanionic component. As used herein, the term “polyanionic component” refers to a chemical entity, for example, an ionically charged species, such as an ionically charged polymeric material, which includes multiple discrete anionic charges. Non-ionic SECs may include polyvinyl alcohol (PVA), polyvinyl pyrrolidone (povidone), and various gums and other non-ionic agents.

In one embodiment, the SEC is a polyanionic component, which may be selected from polymeric materials having multiple anionic charges, and mixtures thereof.

Examples of useful polyanionic components are selected from anionic polymers derived from acrylic acid (meaning to include polymers from acrylic acid, acrylates and the like and mixtures thereof), anionic polymers derived from methacrylic acid (meaning to include polymers from methacrylic acid, methacrylates, and the like and mixtures thereof), anionic polymers derived from alginic acid (meaning to include alginic acid, alginates, and the like and mixtures thereof), anionic polymers of amino acids (meaning to include polymers of amino acids, amino acid salts, and the like and mixtures thereof), and the like, and mixtures thereof. Very useful polyanionic components are those selected from anionic cellulose derivatives and mixtures thereof, especially carboxymethyl cellulose and its derivatives.

A surfactant may be used for assisting in dissolving an excipient or an active agent, dispersing a solid or liquid in a composition, enhancing wetting, modifying drop size, or a number of other purposes. Useful surfactants, include, but are not limited to sorbitan esters, Polysorbate 20, Polysorbate 40, Polysorbate 60, Polysorbate 80, stearates, glyceryl stearate, isopropyl stearate, polyoxyl stearate, propylene glycol stearate, sucrose stearate, polyethylene glycol, polyethylene oxide, polypropylene oxide, polyethylene oxide-polypropylene oxide copolymers, alcohol ethoxylates, alkylphenol ethoxylates, alkyl glycosides, alkyl polyglycosides, fatty alcohols, phospholipids, phosphatidyl choline, phosphatidyl serine, and the like.

Likewise, various useful vehicles may be used in the ophthalmic preparations disclosed herein. These vehicles include, but are not limited to, polyvinyl alcohol, povidone, hydroxypropyl methyl cellulose, poloxamers, carboxymethyl cellulose, hydroxyethyl cellulose and purified water.

Tonicity adjustors may be added as needed or convenient. They include, but are not limited to, salts, particularly sodium chloride, potassium chloride, mannitol and glycerin, or any other suitable ophthalmically acceptable tonicity adjustor.

In a similar vein, an ophthalmically acceptable antioxidant includes, but is not limited to, sodium metabisulfite, sodium thiosulfate, acetylcysteine, butylated hydroxyanisole and butylated hydroxytoluene.

Other excipient components which may be included in the ophthalmic preparations are chelating agents. A useful chelating agent is edetate disodium, although other chelating agents may also be used in place or in conjunction with it.

Compositions may be aqueous solutions or emulsions, or some other acceptable liquid form. For an emulsion, one or more oils will be used to form the emulsion, and in some instances one or more surfactants and/or emulsion stabilization excipients will be required. Suitable oils include, but are not limited to anise oil, castor oil, clove oil, cassia oil, cinnamon oil, almond oil, corn oil, arachis oil, cottonseed oil, safflower oil, maize oil, linseed oil, rapeseed oil, soybean oil, olive oil, caraway oil, rosemary oil, peanut oil, peppermint oil, sunflower oil, eucalyptus oil, sesame oil, and the like.

The composition of the present invention will comprise an effective amount of one or more of the compounds of the above formula. For example the ophthalmic composition may comprise from 0.01% to 10%, preferably 0.1 to 5%, and most preferably from 0.1% to 1%, e.g. 0.2%, by weight, of one or more of the compounds of the above formula.

These and other aspects, objects, and embodiments will be more apparent in the accompanying specific example and drawing FIGURE.

### EXAMPLE

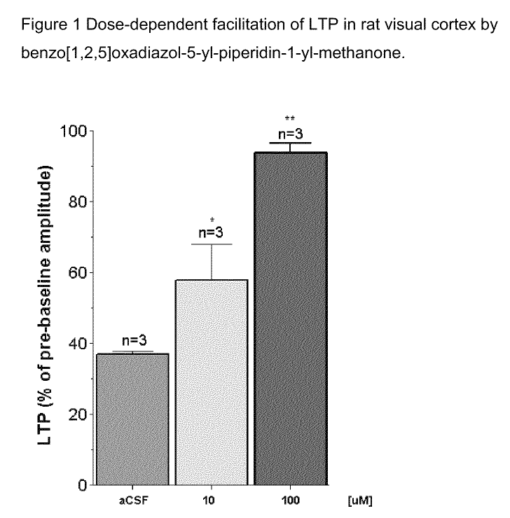

The effect of the Ampakine, i.e. benzo[1,2,5]oxadiazol-5-yl-piperidin-1-yl-methanone, in the visual cortex was investigated using brain slices prepared from primary visual cortex to determine if this compound facilitates the induction of long-term potentiation (LTP). LTP is a well established cellular model for synaptic plasticity and the encoding of information. The visual cortex is one synapse removed from the eye and integrates visual signals generated by the retina. It is thus essential for decoding, processing and transforming visual inputs originating in the eye, and proper visual cortical function is necessary for normal vision. Visual cortex LTP in particular has been demonstrated to have functional consequences on visual evoked responses. It was discovered that the compound produces a marked and dose-dependent enhancement of visual cortex LTP. (See FIG. 1, which shows a dose-dependent facilitation of LTP in rat visual cortex by the compound.)

### Method

### LTP in Visual Cortex Slice

Following decapitation of the anesthetized rat, the brain was rapidly removed and immersed in ice-cold artificial cerebrospinal fluid (ACSF) containing (in mM) NaCl 124, KCl 3, KH₂PO₄ 1.25, CaCl₂ 3.4, MgSO₄ 2.5, NaHCO₃ 26, and D-glucose 10. A block of visual cortex was created by removing the frontal ⅔ portion of the brain and the cerebellum. Coronal visual cortex slices of 350 μm thick were prepared from young adult (200-300 g) male Sprague-Dawley rats using a vibratome (VT 1000 S; Leica). The slices were maintained in an interface recording chamber perfused with preheated ACSF. Slices were continuously perfused with this solution at a rate of 1.00-1.50 ml/min while the surface of the slices was exposed to warm, humidified 95% O₂/5% CO₂ and maintained at 31±1° C. Visual cortex slices were allowed to recover for 1 hr before recording began. A single stimulating and recording electrode was placed in layer IV and III, respectively, to generate and record field excitatory postsynaptic potentials (fEPSPs). Pulses were administered at 0.05 Hz using a current that produced a fEPSP that is 50% of the maximum spike free response. An input-output (IO) curve is done to determine the stimulation needed to achieve a stable baseline. Following a 15 min stable baseline recording period, a train of 5 theta bursts (each burst containing four pulses at 100 Hz with an inter-burst interval of 200 ms) is delivered to the slice. This is repeated 2 additional times with a 1 minute inter-train interval, and the level of LTP was recorded for at least 30 min. Changes in amplitude of the synaptic response were used to measure the extent of LTP, since the amplitude was determined to be the more consistent parameter than the slope of the response. Control LTP values were obtained from slices not treated with drug. Different slices were used to study drug effects on LTP. Drug was infused after 15 min baseline recording for a duration of 20 minutes followed by LTP induction. Drug washout began 5 minutes after tetanization. Recording of the amplitude before, during, and after drug infusion was continuously done at 0.05 Hz. LTP was recorded for at least 30 min after induction.

The present invention is not to be limited in scope by the exemplified embodiments, which are only intended as illustrations of specific aspects of the invention. It will be appreciated that the invention is not limited thereto. In particular, other Ampakines, that act as allosteric upmodulators of AMPA type glutamate receptors in vitro and in vivo will facilitate LTP in the visual cortex. Thus, Ampakines will benefit visual disorders mediated by visual cortex plasticity. In addition, degenerative diseases of the visual system will benefit from Ampakine treatment including retina-based visual dysfunctions (e.g. glaucoma, dry and wet ARM D, geographic atrophy, optic neuritis, rod dystrophies, cone dystrophies, retinopathy, retinitis pigmentosa and other retinopathies) all of which share a common final symptom, i.e., prolonged dysfunctional signal transmission between eye and visual cortex. Accordingly, any and all variations and modifications which may occur to those skilled in the art are to be considered to be within the scope and spirit of the invention as defined in the appended claims

What is claimed is:

1. A method for treating a disorder of the eye, the method comprising the step of administering to a patient in need of such treatment an effective amount of a compound represented by the formula 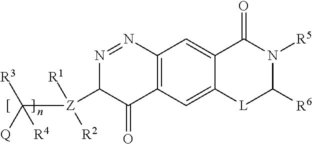
wherein R¹ and R² are independently hydrogen, alkyl, substituted alkyl, cycloalkyl, alkynyl, substituted alkynyl, cyano, alkoxy, carboxamido, substituted carboxamido, and if R¹ and R² are alkyl, R¹ and R² may be joined with a bond or —(CH₂)ₘ— to produce a cycloalkyl, R³ and R⁴ are independently hydrogen, alkyl, hydroxyl, alkoxy, cyano, fluoro, and if R³ and R⁴ are alkyl, R³ and R⁴ may be joined with a bond or —(CH₂)ₘ— to produce a cycloalkyl, Q may be absent, hydrogen, alkyl, cycloalkyl, cycloalkenyl, alkoxy, substituted alkoxy, substituted thio, cyano, thionitrile, sulfonamide, substituted sulfonamide, substituted sulfonyl, aromatic, substituted aromatic, heteroaromatic, substituted heteroaromatic, or bicycloheteroaromatic, R⁵ is hydrogen, alkyl, cycloalkyl, or when R⁶ is also alkyl, together with R⁶ may form a heterocycloalkyl ring, R⁶ may be hydrogen, alkyl, substituted alkyl, or —OR⁷, R⁷ is alkyl or, when R⁵ is alkyl, together with R⁵ forms a 5-, 6-, or 7-membered ring, L may be —O—, —S—, —N═ or absent, Z may be carbon or nitrogen or absent, m is 1, 2 or 3, n is 0, 1 or 2, and when n is 0, Q may be directly bonded to Z; or a pharmaceutically acceptable addition salt thereof.

2. The method of claim 1, wherein the disorder is selected from the group consisting of macular edema, dry and wet macular degeneration, choroidal neovascularization, diabetic retinopathy, acute macular neuroretinopathy, central serous chorioretinopathy, cystoid macular edema, and diabetic macular edema, uveitis, retinitis, choroiditis, acute multifocal placoid pigment epitheliopathy, Behcet's disease, birdshot retinochoroidopathy, syphilis, lyme, tuberculosis, toxoplasmosis, intermediate uveitis (pars planitis), multifocal choroiditis, multiple evanescent white dot syndrome (mewds), ocular sarcoidosis, posterior scleritis, serpiginous choroiditis, subretinal fibrosis and uveitis syndrome, Vogt-Koyanagi-and Harada syndrome; retinal arterial occlusive disease, anterior uveitis, retinal vein occlusion, central retinal vein occlusion, disseminated intravascular coagulopathy, branch retinal vein occlusion, hypertensive fundus changes, ocular ischemic syndrome, retinal arterial microaneurysms, Coat's disease, parafoveal telangiectasis, hemiretinal vein occlusion, papillophlebitis, central retinal artery occlusion, branch retinal artery occlusion, carotid artery disease (CAD), frosted branch angiitis, sickle cell retinopathy, angioid streaks, familial exudative vitreoretinopathy, and Eales disease; traumatic/surgical conditions such as sympathetic ophthalmia, uveitic retinal disease, retinal detachment, trauma, photocoagulation, hypoperfusion during surgery, radiation retinopathy, and bone marrow transplant retinopathy; proliferative vitreal retinopathy and epiretinal membranes, and proliferative diabetic retinopathy; infectious disorders such as ocular histoplasmosis, ocular toxocariasis, presumed ocular histoplasmosis syndrome (PONS), endophthalmitis, toxoplasmosis, retinal diseases associated with HIV infection, choroidal disease associate with HIV infection, uveitic disease associate with HIV infection, viral retinitis, acute retinal necrosis, progressive outer retinal necrosis, fungal retinal diseases, ocular syphilis, ocular tuberculosis, diffuse unilateral subacute neuroretinitis, and myiasis; genetic disorders such as retinitis pigmentosa, systemic disorders with associated retinal dystrophies, congenital stationary night blindness, cone dystrophies, Stargardt's disease and fundus flavimaculatus, Best's disease, pattern dystrophy of the retinal pigmented epithelium, X-linked retinoschisis, Sorsby's fundus dystrophy, benign concentric maculopathy, Bietti's crystalline dystrophy, and pseudoxanthoma elasticum; retinal tears/holes such as retinal detachment, macular hole, and giant retinal tear; tumors such as retinal disease associated with tumors, congenital hypertrophy of the retinal pigmented epithelium, posterior uveal melanoma, choroidal hemangioma, choroidal osteoma, choroidal metastasis, combined hamartoma of the retina and retinal pigmented epithelium, retinoblastoma, vasoproliferative tumors of the ocular fundus, retinal astrocytoma, and intraocular lymphoid tumors; punctate inner choroidopathy, acute posterior multifocal placoid pigment epitheliopathy, myopic retinal degeneration, acute retinal pigment epitheliitis, retinitis pigmentosa, proliferative vitreal retinopathy (PVR), age-related macular degeneration (ARMD), diabetic retinopathy, diabetic macular edema, retinal detachment, retinal tear, uveitus, cytomegalovirus retinitis, and glaucoma.

3. A method for treating a visual disorder mediated by the visual cortex comprising the step of administering to a patient in need of such treatment a compound 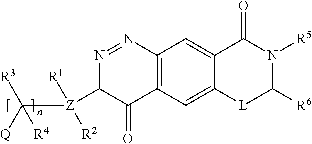
Wherein R¹ and R² are independently hydrogen, alkyl, substituted alkyl, cycloalkyl, alkynyl, substituted alkynyl, cyano, alkoxy, carboxamido, substituted carboxamido, and if R¹ and R² are alkyl, R¹ and R² may be joined with a bond or —(CH₂)ₘ— to produce a cycloalkyl, R³ and R⁴ are independently hydrogen, alkyl, hydroxyl, alkoxy, cyano, fluoro, and if R³ and R⁴ are alkyl, R³ and R⁴ may be joined with a bond or —(CH₂)ₘ— to produce a cycloalkyl, Q may be absent, hydrogen, alkyl, cycloalkyl, cycloalkenyl, alkoxy, substituted alkoxy, substituted thio, cyano, thionitrile, sulfonamide, substituted sulfonamide, substituted sulfonyl, aromatic, substituted aromatic, heteroaromatic, substituted heteroaromatic, or bicycloheteroaromatic, R⁵ is hydrogen, alkyl, cycloalkyl, or when R⁶ is also alkyl, together with R⁶ may form a heterocycloalkyl ring, R⁶ may be hydrogen, alkyl, substituted alkyl, or —OR⁷, R⁷ is alkyl or, when R⁵ is alkyl, together with R⁵ forms a 5-, 6-, or 7-membered ring, L may be —O—, —S—, —N═ or absent, Z may be carbon or nitrogen or absent, m is 1, 2 or 3, n is 0, 1 or 2, and when n is 0, Q may be directly bonded to Z, or a pharmaceutically acceptable addition salt thereof.

4. The method of claim 3, wherein the disorder is selected from the group consisting of amblyopia, stroke-induced blindness, visual dysfunction associated with Parkinson's disease and Alzheimer's disease, seizure-induced cortical blindness, mild traumatic brain injury-induced visual dysfunction, and epileptic blindness.

5. The method of claim 3 further comprising enhancing the induction of long-term potentiation in the primary visual cortex (V1).

6. The method of claim 5 wherein said disorder is selected from the group consisting of amblyopia, stroke-induced blindness, visual dysfunction in Parkinson's disease and Alzheimer's disease, seizure-induced cortical blindness, induced visual dysfunction, and epileptic blindness.

7. An ophthalmic composition comprising a compound represented by the formula 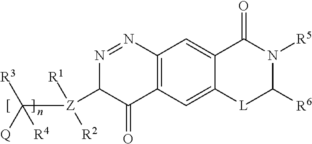
Wherein R¹ and R² are independently hydrogen, alkyl, substituted alkyl, cycloalkyl, alkynyl, substituted alkynyl, cyano, alkoxy, carboxamido, substituted carboxamido, and if R¹ and R² are alkyl, R¹ and R² may be joined with a bond or —(CH₂)ₘ— to produce a cycloalkyl, R³ and R⁴ are independently hydrogen, alkyl, hydroxyl, alkoxy, cyano, fluoro, and if R³ and R⁴ are alkyl, R³ and R⁴ may be joined with a bond or —(CH₂)ₘ— to produce a cycloalkyl, Q may be absent, hydrogen, alkyl, cycloalkyl, cycloalkenyl, alkoxy, substituted alkoxy, substituted thio, cyano, thionitrile, sulfonamide, substituted sulfonamide, substituted sulfonyl, aromatic, substituted aromatic, heteroaromatic, substituted heteroaromatic, or bicycloheteroaromatic, R⁵ is hydrogen, alkyl, cycloalkyl, or when R⁶ is also alkyl, together with R⁶ may form a heterocycloalkyl ring, R⁶ may be hydrogen, alkyl, substituted alkyl, or —OR⁷, R⁷ is alkyl or, when R⁵ is alkyl, together with R⁵ forms a 5-, 6-, or 7-membered ring, L may be —O—, —S—, —N═ or absent, Z may be carbon or nitrogen or absent, m is 1, 2 or 3, n is 0, 1 or 2, and when n is 0, Q may be directly bonded to Z; or a pharmaceutically acceptable addition salt thereof. In combination with an ophthalmically-acceptable carrier or vehicle.

8. The method of claim 1 wherein said compound is benzo[1,2,5]oxadiazol-5-yl-piperidin-1-yl-methanone.

9. The method of claim 2 wherein said compound is benzo[1,2,5]oxadiazol-5-yl-piperidin-1-yl-methanone.

10. The method of claim 3 wherein said compound is benzo[1,2,5]oxadiazol-5-yl-piperidin-1-yl-methanone.

11. The method of claim 4 wherein said compound is benzo[1,2,5]oxadiazol-5-yl-piperidin-1-yl-methanone.

12. The method of claim 5 wherein said compound is benzo[1,2,5]oxadiazol-5-yl-piperidin-1-yl-methanone.

13. The method of claim 6 wherein said compound is benzo[1,2,5]oxadiazol-5-yl-piperidin-1-yl-methanone.

14. The composition of claim 7 wherein said compound is benzo[1,2,5]oxadiazol-5-yl-piperidin-1-yl-methanone.

15. The composition of claim 7 wherein said vehicle is a physiological saline solution.

16. The composition of claim 7 further comprising a buffer and one or more pharmaceutically acceptable preservative, stabilizer and/or surfactant.

17. The composition of claim 16 wherein said buffer is selected from the group consisting of acetate buffers, citrate buffers, phosphate buffers and borate buffers.

18. The composition of claim 16 wherein said preservative is selected from the group consisting of benzalkonium chloride, chlorobutanol, thimerosal, phenylmercuric acetate and phenylmercuric nitrate.

19. The composition of claim 16 wherein said surfactant is selected from the group consisting of sorbitan esters, Polysorbate 20, Polysorbate 40, Polysorbate 60, Polysorbate 80, stearates, glyceryl stearate, isopropyl stearate, polyoxyl stearate, propylene glycol stearate, sucrose stearate, polyethylene glycol, polyethylene oxide, polypropylene oxide, polyethylene oxide-polypropylene oxide copolymers, alcohol ethoxylates, alkylphenol ethoxylates, alkyl glycosides, alkyl polyglycosides, fatty alcohols, phospholipids, phosphatidyl choline and phosphatidyl serine.

20. The composition of claim 7 wherein said vehicle is selected from the group consisting of polyvinyl alcohol, povidone, hydroxypropyl methyl cellulose, poloxamers, carboxymethyl cellulose, hydroxyethyl cellulose and purified water.

21. The composition of claim 7 further comprising a tonicity adjuster selected from the group consisting of sodium chloride, potassium chloride, mannitol and glycerin.

22. The composition of claim 7 further comprising an antioxidant selected from the group consisting of sodium metabisulfite, sodium thiosulfate, acetylcysteine, butylated hydroxyanisole and butylated hydroxytoluene.

23. The composition of claim 7 further comprising a chelating agent wherein said chelating agent is edetate disodium

24. The composition of claim 7 wherein said compound comprises from about 0.01% to 10%.

25. The composition of claim 7 further comprising a solubility enhancing component (SEC).

26. The composition of claim 25 wherein said SEC is an anionic cellulose derivative.

27. The composition of claim 26 wherein said SEC is a carboxymethyl cellulose or derivative thereof.

28. The composition of claim 7 wherein said composition is an emulsion and said vehicle is an oil.

29. The composition of claim 28 wherein said oil is selected from the group consisting of anise oil, castor oil, clove oil, cassia oil, cinnamon oil, almond oil, corn oil, arachis oil, cottonseed oil, safflower oil, maize oil, linseed oil, rapeseed oil, soybean oil, olive oil, caraway oil, rosemary oil, peanut oil, peppermint oil, sunflower oil, eucalyptus oil and sesame oil.
